(19) United States
(12) Patent Application Publication
(10) Pub. No.: US 2008/0071152 A1
(43) Pub. Date: Mar. 20, 2008

(54) AUTONOMIC-NERVOUS STATE JUDGING DEVICE, AUTONOMIC-NERVOUS STATE JUDGING METHOD, AND COMPUTER PROGRAM PRODUCT

(75) Inventors: Akihisa MORIYA, Tokyo (JP); Takuji SUZUKI, Kanagawa (JP)

Correspondence Address: OBLON, SPIVAK, MCCLELLAND MAIER & NEUSTADT, P.C., 1940 DUKE STREET, ALEXANDRIA, VA, 22314, US

(73) Assignee: Kabushiki Kaisha Toshiba, Tokyo (JP)

(21) Appl. No.: 11/854,935

(22) Filed: Sep. 13, 2007

(30) Foreign Application Priority Data
Sep. 14, 2006 (JP) 2006-250059
Aug. 13, 2007 (JP) 2007-210601

(51) Int. Cl. A61B 5/00

(52) U.S. Cl. 600/301

(57) ABSTRACT

An autonomic-nervous state judging device measures an autonomic-nervous index of a subject in sleep at predetermined time intervals. The autonomic-nervous state judging device sets a plurality of cycle frames of a predetermined length each including one sleep cycle, and a judging frame in a cycle frame at a predetermined position. The autonomic-nervous state judging device specifies state of autonomic nervous system in the judging frame based on the autonomic-nervous index, and detects timing of a change in the state of the autonomic nervous system.

### CROSS-REFERENCE TO RELATED APPLICATIONS

This application is based upon and claims the benefit of priority from the prior Japanese Patent Application No. 2006-250059 filed on Sep. 14, 2006 and the prior Japanese Patent Application No. 2007-210601 filed on Aug. 13, 2007; the entire contents of which are incorporated herein by reference.

### BACKGROUND OF THE INVENTION

1. Field of the Invention

The present invention relates to an autonomic-nervous state judging device, an autonomic-nervous state judging method, and a computer program product.

2. Description of the Related Art

To measure stages of sleep, there is a known technology in which brain waves, eyeball movement, electromyographic activity and the like are recorded by using sleep polygraphs. There is another known technology in which pulse fluctuation is analyzed based on pulse intervals to determine the state of autonomic nervous system. For example, JP-A H7-143972 (KOKAI) discloses a technology for judging sleep state based on sympathetic and parasympathetic activities. JP-A 2005-152310 (KOKAI) discloses a technology in which the sleep stage of a subject is detected in real time by using information such as pulse fluctuation of the subject lying on an air mattress.

It is said that if a person is woken up at the later stage of rapid eye movement (REM) sleep, the person feels fresh. Therefore, it is preferred that a person be awakened at the later stage of REM sleep. Thus, there is a demand for a technology of controlling awakening time according to the state of autonomic nervous system such as sleep state.

In such awakening control, it is necessary to specify timing of a change in the state of autonomic nervous system. However, the state of autonomic nervous system substantially fluctuates, and it is difficult to accurately specify timing of a change in its state.

### SUMMARY OF THE INVENTION

According to an aspect of the present invention, an autonomic-nervous state judging device includes a measuring unit that measures, at regular intervals, an autonomic-nervous index that indicates state of autonomic nervous system of a subject in a sleep state, a first setting unit that sets a plurality of cycle frames of a first time length, each of which includes one sleep cycle, at intervals shorter than the first time length, a second setting unit that sets a judging frame of a second time length shorter than the first time length at a predetermined position in each of the cycle frames, a specifying unit that specifies state of autonomic nervous system in the judging frame based on the autonomic-nervous index in corresponding one of the cycle frames, and a detecting unit that detects time of a change in the state of autonomic nervous system specified by the specifying unit.

According to another aspect of the present invention, an autonomic-nervous state judging method includes measuring, at regular intervals, an autonomic-nervous index that indicates state of autonomic nervous system of a subject in a sleep state, setting a plurality of cycle frames of a first time length, each of which includes one sleep cycle, at intervals shorter than the first time length, setting a judging frame of a second time length shorter than the first time length at a predetermined position in each of the cycle frames, specifying unit state of autonomic nervous system in the judging frame based on the autonomic-nervous index in corresponding one of the cycle frames, and detecting time of a change in the state of autonomic nervous system specified at the specifying.

According to still another aspect of the present invention, a computer program product includes a computer program that implements the above methods on a computer.

### BRIEF DESCRIPTION OF THE DRAWINGS

FIG. 1 is a block diagram of an autonomic-nervous state judging system according to a first embodiment of the present invention;

FIG. 2 is an example of how an autonomic-nervous state judging device is attached to a subject;

FIG. 3 is a schematic diagram for explaining a process performed by an autonomic-nervous index calculator shown in FIG. 1;

FIG. 4 is a graph for explaining a process performed by a cycle frame setting unit shown in FIG. 1;

FIG. 5 is a schematic diagram for explaining a judging frame;

FIG. 6 is a graph of autonomic-nervous indices obtained during a sleep state of a subject;

FIG. 7 is a flowchart of a process procedure of awakening a subject according to the first embodiment;

FIG. 8 is a detailed flowchart of a sleep-state judgment process shown in FIG. 7;

FIG. 9 is a detailed flowchart of an awakening-time control process shown in FIG. 7;

FIG. 10 is a schematic for explaining a process performed by a change-timing detecting unit shown in FIG. 1;

FIG. 11 is a block diagram of a hardware configuration of the autonomic-nervous state judging device;

FIG. 12 is a block diagram of an autonomic-nervous state judging system according to a second embodiment of the present invention;

FIG. 13 is a flowchart of a process procedure of awakening a subject according to the second embodiment;

FIG. 14 is a detailed flowchart of an predominance judgment process shown in FIG. 13; and

FIG. 15 is a detailed flowchart of an awakening-time control process shown in FIG. 13.

### DETAILED DESCRIPTION OF THE INVENTION

Exemplary embodiments of the present invention are explained in detail below with reference to the accompanying drawings.

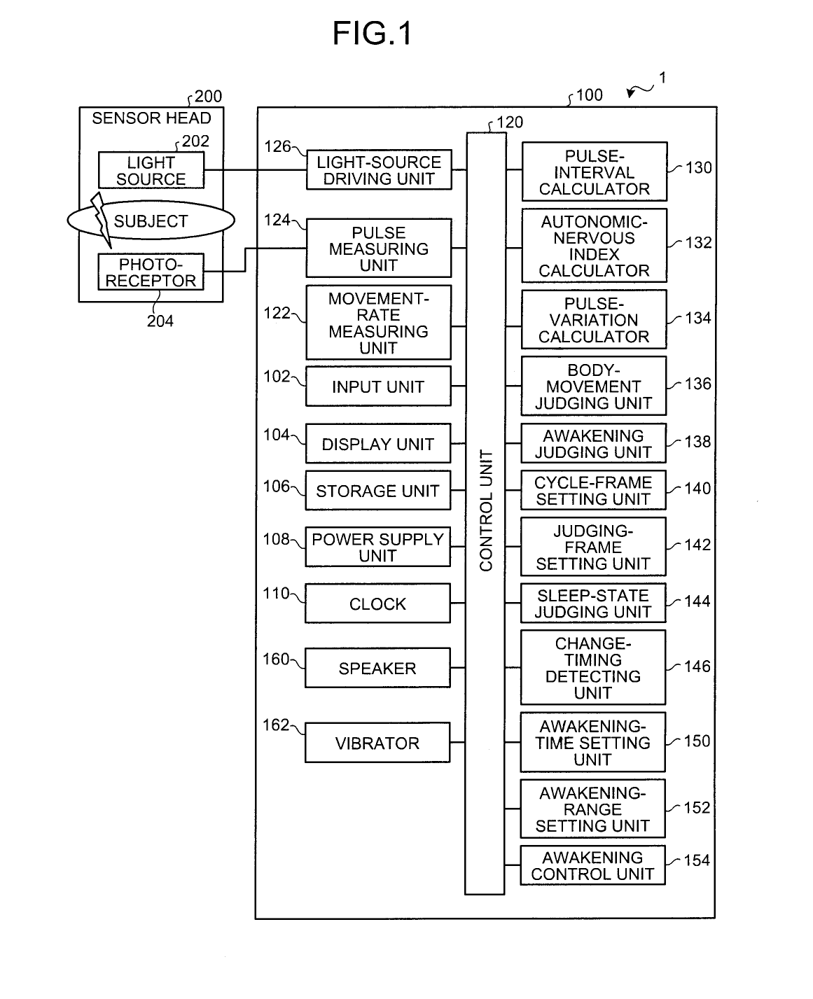

FIG. 1 is a block diagram of an autonomic-nervous state judging system 1 according to a first embodiment of the present invention. The autonomic-nervous state judging system 1 detects timing of changes in a sleep state of a subject, and awakens a subject based on the changes.

The autonomic-nervous state judging system 1 includes an autonomic-nervous state judging device 100 and a sensor head 200. The autonomic-nervous state judging device 100 includes an input unit 102, a display unit 104, a storage unit 106, a power supply unit 108, a clock 110, a control unit 120, a movement-rate measuring unit 122, a pulse measuring unit 124, a light-source driving unit 126, a pulse-interval calculator 130, an autonomic-nervous index calculator 132, a pulse-variation calculator 134, a body-movement judging unit 136, an awakening judging unit 138, a cycle-frame setting unit 140, a judging-frame setting unit 142, a sleep-state judging unit 144, a change-timing detecting unit 146, an awakening-time setting unit 150, an awakening-range setting unit 152, an awakening control unit 154, a speaker 160, and a vibrator 162. The sensor head 200 includes a light source 202 and a photoreceptor 204.

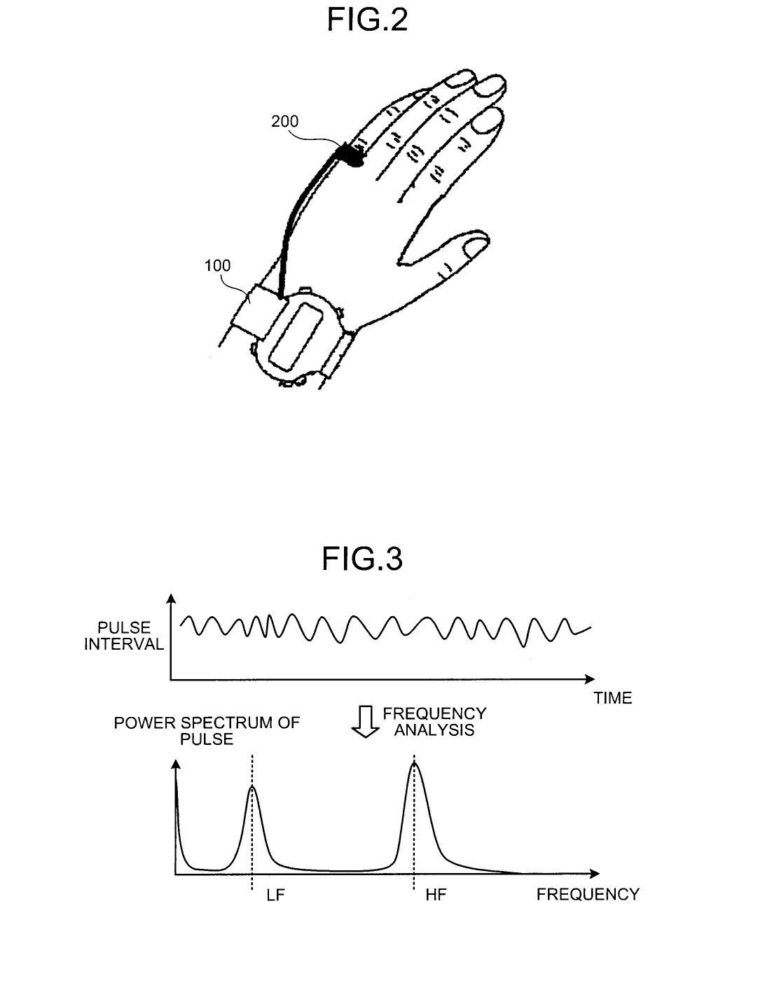

FIG. 2 is an example of how the autonomic-nervous state judging device 100 is attached to a subject. The autonomic-nervous state judging device 100 can be worn on a wrist like a wristwatch, and in this case, the sensor head 200 for measuring pulse is worn, for example, on a little finger.

The input unit 102 is a switch that is used by a user to switch the power ON/OFF and to provide requests or instructions to change display. The display unit 104 includes, for example, a liquid crystal display (LCD), and displays various types of information such as a result of sleep state judgment.

The storage unit 106 stores therein measurement data such as pulse data, electrocardiographic data, and body movement data. The storage unit 106 further stores therein data that are obtained after various processes such as pulse interval data, and pulse variation data, and a threshold value used to judge the state of sleep. The storage unit 106 can be a flash memory or the like. The power supply unit 108 is a battery that supplies power to the autonomic-nervous state judging device 100. The clock 110 is a timer, specifically, a real-time clock integrated circuit and the like.

The control unit 120 controls measuring time, and stores and processes received data. The movement-rate measuring unit 122 measures the rate of body movement to obtain analog movement rate data and converts the analog data to digital data. The movement-rate measuring unit 122 is a sensor that senses body movements in three axial directions of −2 g to 2 g, and is mounted on the autonomic-nervous state judging device 100. Specifically, the analog data is converted through a 10-bit analog-to-digital (A/D) converter to digital data after adjustment of gain and offset of the analog data through an adjusting circuit. The movement-rate measuring unit 122 outputs the digital data to the control unit 120.

The sensor head 200 includes, for example, a blue light emitting diode as the light source 202 and a photo diode as the photoreceptor 204. The sensor head 200 irradiates the skin surface of a subject with light, and detects changes that occur in reflected light due to changes in blood flow in capillaries.

The pulse measuring unit 124 measures pulse of a subject to obtain analog data, and converts the analog data to digital data. Specifically, the pulse measuring unit 124 converts electric current output from the photo diode of a pulse sensor, that is, the sensor head 200, into voltage through a current-to-voltage converter. The pulse measuring unit 124 amplifies the voltage with an amplifier, and, after filtering it with a high-pass filter (cutoff frequency: 0.1 Hz) and a low-pass filter (cutoff frequency: 50 Hz), converts the analog data to digital data through the 10-bit A/D converter. Pulse data after conversion is output to the control unit 120. The light-source driving unit 126 drives the light source 202. The pulse-interval calculator 130 calculates pulse interval data from the pulse data obtained by the pulse measuring unit 124. The pulse-interval data is data on time intervals in one pulse cycle. Specifically, the pulse-interval calculator 130 takes a sample of pulse data from the pulse measured by the pulse measuring unit 124. The pulse-interval calculator 130 differentiates the sampled pulse data with respect to time to derive direct current (DC) fluctuation component in the pulse data, and eliminates the DC fluctuation component from the pulse data.

After the removal of the DC fluctuation component, the pulse-interval calculator 130 acquires the maximum value and the minimum value of the pulse data during a period of approximately one second before and after a processing point in the pulse data. A value between the maximum value and the minimum value is set as a pulse interval threshold. For example, with a difference between the maximum value and the minimum value defined as amplitude, a value of 90% of the amplitude from the minimum value is used as the pulse interval threshold. The pulse-interval calculator 130 also detects the time when a sequence of pulses with an interval that matches the pulse interval threshold appears, and obtains pulse interval data.

The pulse interval data is unequally spaced data, and to be converted to an equally spaced data for performing frequency analysis. The pulse-interval calculator 130 performs interpolation of the unequally spaced data, and performs resampling to generate equally spaced pulse interval data. For example, the pulse-interval calculator 130 generates equally spaced pulse interval data by using three sampling points around a point of interpolation according to a third-order polynomial interpolation technique.

The autonomic-nervous index calculator 132 calculates two autonomic-nervous indices for judging a sleep state of a subject. The two autonomic-nervous indices are a low-frequency (LF) index in a low-frequency area of about 0.05 Hz to 0.15 Hz and a high-frequency (HF) index in a high-frequency area of about 0.15 Hz to 0.4 Hz.

FIG. 3 depicts graphs for explaining a process performed by the autonomic-nervous index calculator 132, in which equally spaced pulse interval data is converted into a frequency spectral distribution through fast Fourier transform (FFT) method. The autonomic-nervous index calculator 132 acquires the autonomic-nervous indices LF and HF from the frequency spectral distribution. Specifically, the autonomic-nervous index calculator 132 acquires the autonomic-nervous indices LF and HF by taking arithmetic average of total of three points, i.e., a peak value of a plurality of power spectra, and points before and after the peak value at even intervals from the peak value.

From the point of view of reducing load of data processing, the FFT method is used in the embodiment as a frequency analyzing method. However, other methods such as autoregressive (AR) model, maximum entropy method, wavelet method can be used.

The pulse-variation calculator 134 calculates pulse variation in the pulse data obtained by the pulse measuring unit 124. The pulse variation is a second-by-second variation in the pulse of a subject within one minute. The body-movement judging unit 136 differentiates the three-axial direction movement rate data obtained by the movement-rate measuring unit 122, with respect to time, and obtains a differential coefficient thereof. The body-movement judging unit 136 obtains variation in body movement and the amount of body movement. The variation in body movement is represented by a square-root of sum of squares of the differential coefficient of each movement rate in three axial directions. The amount of body movement is represented by an average of variations in body movement during a pulse interval. When the variation in body movement is larger than a predetermined threshold, the body-movement judging unit 136 judges that body movement has occurred. For example, the minimum value of 0.01 G (gravity), used to measure minute body movement, is used as the predetermined threshold.

The awakening judging unit 138 judges that a subject is awake when frequency of occurrence of body movement is equal to or higher than a predetermined threshold. The awakening judging unit 138 judges that a subject is sleeping when frequency of occurrence of body movement is lower than the predetermined threshold. For example, from frequency of occurrence of body movement in the past awakening state, 20 times/minute is preferably used as the predetermined threshold. Specifically, the awakening judging unit 138 acquires information about occurrence of body movement from the body-movement judging unit 136, and calculates frequency of occurrence of body movement in a set interval zone.

The cycle-frame setting unit 140 sets a cycle frame, that is, a time interval or a time frame including one cycle of sleep (hereinafter, “sleep cycle”). Length of the one sleep cycle is about 90 minutes to 120 minutes. Accordingly, the cycle-frame setting unit 140 can set the cycle frame to, for example, 120 minutes or 90 minutes. The length of the cycle frame is cited above by way of example, and the cycle frame can be set to any length as long as it includes the sleep cycle. In the following example, the cycle-frame setting unit 140 sets a time period of past 120 minutes from the current time as a cycle frame.

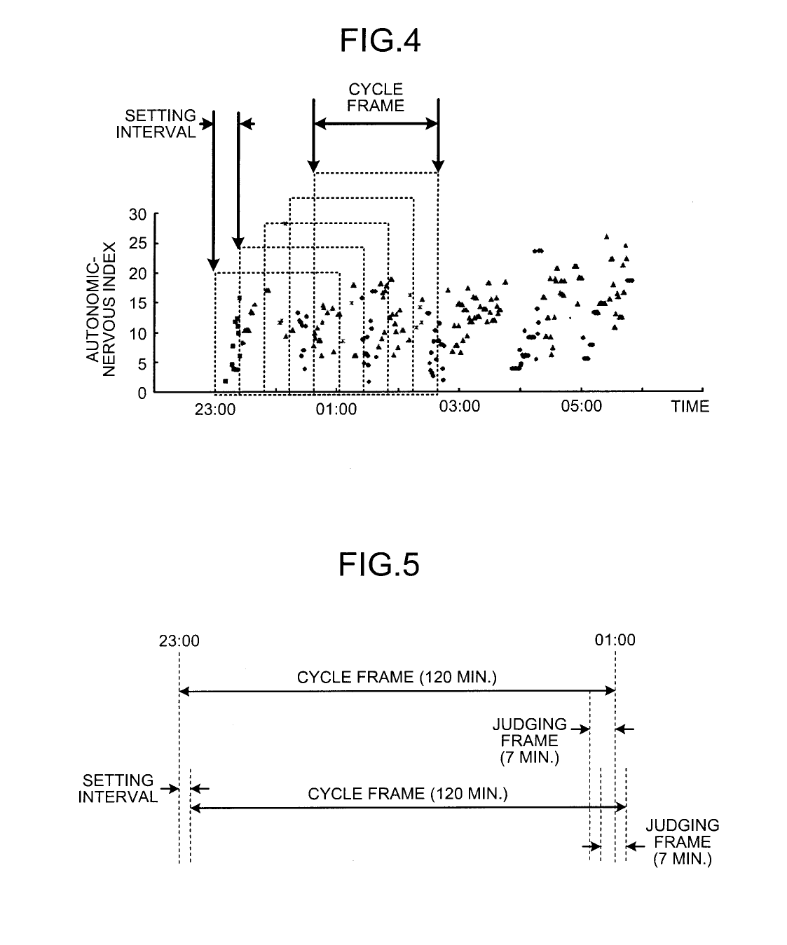

As shown in FIG. 4, if measuring of the autonomic-nervous index starts at 23:00 o'clock, a time period from 23:00 o'clock to 01:00 o'clock is set as a cycle frame. During the time period of the cycle frame autonomic-nervous indices are measured continuously. The cycle frame setting unit 140 sets cycle frames at a setting interval of, for example, one minute. Specifically, at 01:01 o'clock, the cycle frame setting unit 140 sets a time period of 120 minutes from 23:01 o'clock to 01:01 o'clock as a cycle frame. The setting interval can be set to any length as long as it is shorter than the cycle frame. It is preferred that the setting interval be shorter as compared to a judging frame, which is described at a later stage.

Referring back to FIG. 1, the judging-frame setting unit 142 sets a judging frame, which is a time frame to judge a change in sleep state of a subject. A time length of the judging frame is set in advance, and the judging frame is, for example, a rearmost portion of the cycle frame.

In an example of FIG. 5, a judging frame is set in a cycle frame from 23:00 o'clock to 01:00 o'clock. That is, the judging frame is a time frame of seven minutes ending at 01:00 o'clock. A judging frame set in a cycle frame from 23:01 o'clock to 01:01 o'clock is of a time frame of seven minutes ending at 01:01 o'clock. Thus, a relative position of a judging frame in a cycle frame is set in advance. When a cycle frame is set, an absolute position of a judging frame is specified based on the relative position.

For example, when a judging frame of seven minutes is to be set at the rearmost portion of a cycle frame, and when the cycle-frame setting unit 140 sets a cycle frame from 23:00 o'clock to 01:00 o'clock, the judging-frame setting unit 142 sets a judging frame from 00:53 o'clock to 01:00 o'clock. The judging frame is only required to be a time frame whose relative position in a cycle frame is set in advance, and need not be a time frame that is at a rearmost portion of the cycle frame.

When the cycle frame setting unit 140 sets cycle frames at a setting interval of one minute, the judging-frame setting unit 142 sets judging frames in the cycle frames at one minute time interval.

The sleep-state judging unit 144 judges a sleep state of a subject as state of autonomic nervous system of the subject based on autonomic-nervous indices LF and HF calculated by the autonomic-nervous index calculator 132 and pulse variation calculated by the pulse-variation calculator 134. As the sleep state, depth of sleep is calculated. The depth of sleep is an index indicating brain activity of a subject. In the first embodiment, the sleep-state judging unit 144 judges a sleep state of a subject based on non-rapid eye movement (NREM) sleep or rapid eye-movement (REM) sleep.

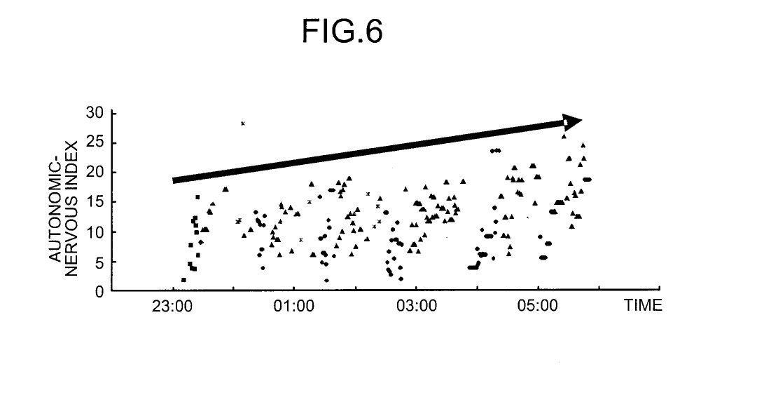

REM sleep or NREM sleep, as shown in FIG. 6, is differentiated according to a magnitude correlation between the autonomic-nervous indices and a threshold. In an example shown in FIG. 6, there is a gradual increase in values of the autonomic-nervous indices corresponding to REM sleep and NREM sleep as time passes. This happens due to circadian rhythm. When a base of the autonomic-nervous indices increases, if all autonomic-nervous indices are used to judge whether a subject is in a state of REM sleep or NREM sleep, there is high possibility of misjudgment.

In addition, the autonomic-nerve indices are likely to vary according to an individual. Therefore, if values of autonomic-nervous indices during a time period from 23:00 o'clock to 05:00 o'clock are used to judge a sleep state of a subject, it is not possible to determine the sleep state accurately.

According to the embodiment, the sleep-state judging unit 144 judges sleep state at a plurality of points of time using autonomic-nervous indices only from a judging frame in a cycle frame. Thus, influence of the circadian rhythm is eliminated, and it is possible to judge sleep state accurately.

The change-timing detecting unit 146 detects timing of a change in sleep state based on sleep state in each of judging frames judged by the sleep-state judging unit 144.

The awakening-time setting unit 150 sets awakening time according to an instruction from a subject. The awakening-range setting unit 152 sets an awakening time range. The term “awakening time range” as used herein refers to a range of a time period predetermined based on the awakening time. The awakening time range is, for example, two hours.

More specifically, the awakening-range setting unit 152 holds a predetermined range of time period, and determines the predetermined range as the awakening time range based on the awakening time set by the awakening-time setting unit 150.

In the embodiment, the awakening-range setting unit 152 determines the awakening time range to a predetermined time period earlier than the awakening time set by the awakening-time setting unit 150. However, the awakening time range is not limited to the above-mentioned range.

It is possible to set the awakening time range to a predetermined time period later than the awakening time. It is also possible to set the awakening time range to a predetermined time period with the awakening time at the middle of the time period.

The awakening control unit 154 determines time to start the speaker 160 and the vibrator 162, and starts the speaker 160 and the vibrator 162 at the time. The speaker 160 outputs alarm sound, and the vibrator 162 generates vibrations to awaken a subject. Thus, the awakening control unit 154 controls an environment around a subject by starting the speaker 160 and the vibrator 162 at the awakening time.

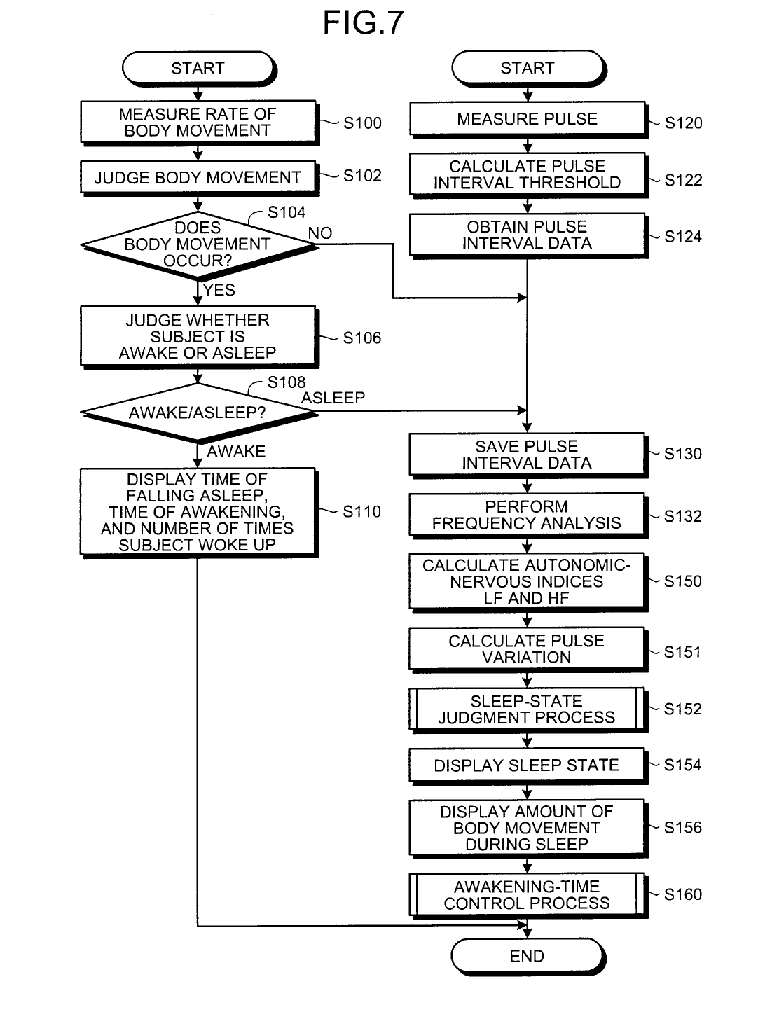

FIG. 7 is a flowchart of an awakening process performed by the autonomic-nervous state judging system 1. A subject wears the autonomic-nerve condition judging system 1 before falling asleep, and switches it ON to start up the awakening function through the input unit 102. The subject specifies a time. Accordingly, the awakening-time setting unit 150 sets the time as awakening time. The movement-rate measuring unit 122 starts measuring the rate of body movement (step S100). The pulse measuring unit 124 starts measuring the pulse (step S120).

The movement-rate measuring unit 122 measures the rate of body movement to obtain movement rate data that indicates body movements in three axial directions. The body-movement judging unit 136 obtains body movement data from the movement rate data acquired by the movement-rate measuring unit 122. When the variation in body movement is larger than a predetermined threshold, the body-movement judging unit 136 judges that body movement has occurred (step S102). When body movement has occurred (Yes at step S104), the awakening judging unit 138 judges whether the subject is awake or asleep (step S106). When the subject is awake (Awake at step S108), the awakening judging unit 138 causes the storage unit 106 to store therein time of falling asleep, awakening time and the number of times the subject woke up during sleep. The awakening judging unit 138 also displays the time of falling asleep, the awakening time and the number of times the subject woke up during sleep on the display unit 104 (step S110).

On the other hand, when the pulse measuring unit 124 starts measuring the pulse, the pulse-interval calculator 130 calculates a pulse interval threshold, which is a dynamic threshold for calculating a pulse interval (step S122). From pulse data in which DC fluctuation component has been eliminated, the pulse-interval calculator 130 detects the time when a sequence of pulses with an interval that matches the pulse interval threshold appears, and obtains pulse interval data (step S124).

The pulse-interval calculator 130 saves the pulse interval data (step S130) only when there is no body movement (No at step S104) and the subject is in a state of sleep (Sleep at step S108).

The pulse-interval calculator 130 converts the pulse interval data to a frequency spectral distribution according to a frequency analysis method such as FFT method (step S132). The autonomic-nervous index calculator 132 calculates autonomic-nervous indices LF and HF from a plurality of power spectra values in the pulse interval data that has been converted into a frequency spectral distribution at step S132 (step S150). The pulse-variation calculator 134 calculates pulse variation (step S151). The autonomic-nervous indices LF and HF, and the pulse variation are associated with time of their detection and stored in the storage unit 106.

The sleep-state judging unit 144 performs a sleep-state judgment process (step S152), i.e., judges sleep state based on the autonomic-nervous indices LF and HF and the pulse variation. The sleep-state judging unit 144 stores the sleep state associated with time of detection in the storage unit 106. The display unit 104 displays the sleep state (step S154), and amount of body movement during sleep (step S156). The awakening control unit 154 performs awakening-time control process (step S160), and the process ends.

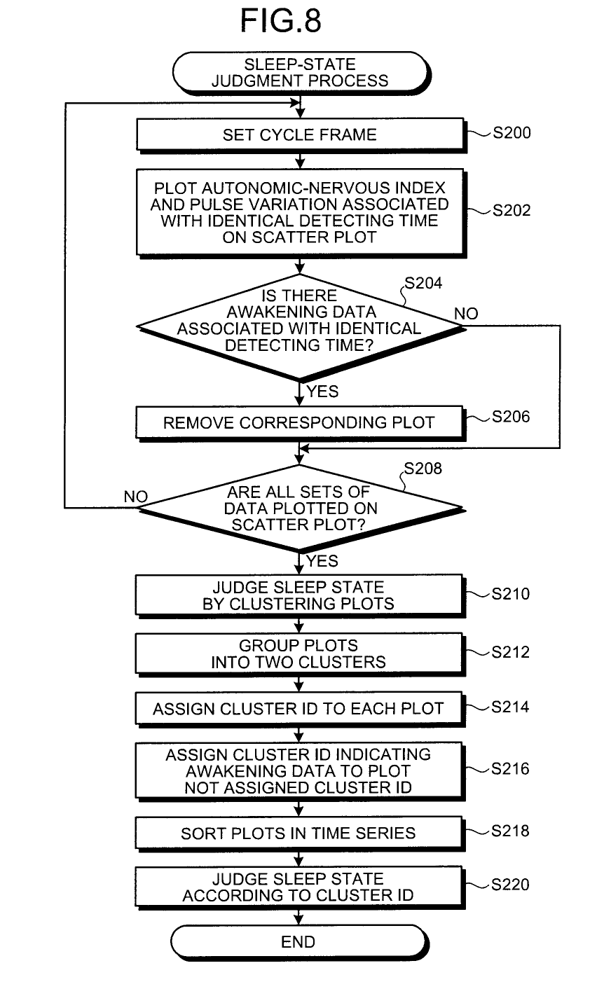

The sleep-state judgment process performed at step S152 of FIG. 7 is explained in detail with reference to FIG. 8. The cycle-frame setting unit 140 sets a cycle frame (step S200). The cycle-frame according to the embodiment is of 120 minutes. Therefore process procedure from step S202 to step S206 is performed with respect to data obtained during a period of 120 minutes.

The autonomic-nervous indices LF and HF obtained at step S150 and the pulse variation obtained at step S151 each stored in association with an identical detection time are plotted in plane coordinates of a scatter plot (step S202). If the storage unit 106 stores therein awakening data associated with a detection time identical to the detection time corresponding to the plot (Yes at step S204), the plot is removed from the scatter plot (step S206). Thus, it is possible to judge sleep state of the subject from the data obtained only when the subject is in a state of sleep. Therefore, sleep state can be judged more accurately.

In the plane coordinate, X coordinate represents LF/HF, and Y coordinate represents pulse variation. It is also possible that X coordinate represents LF and Y coordinate represents HF. Steps S202 to S206 are repeated until all data are plotted on the scatter plot (Yes at step S208). In the embodiment, 120 sets of data (data plots) are plotted on the scatter plot.

The state of sleep is judged by clustering the data plots on the scatter plot (step S210). Specifically, the data plots are grouped into two clusters using K-means algorithm (step S212). A cluster whose center is closer to the origin of the plane coordinate is assigned cluster identifier (ID) 1 as a first cluster. A cluster whose center is away from the origin of the plane coordinate is assigned cluster ID 2 as a second cluster. K-means algorithm is used in the embodiment for clustering to reduce load of data processing. However, it is possible to use other methods such as fuzzy c-means (FCM) method, and entropy method.

A cluster ID is assigned to data plotted on the scatter plot (step S214). A data plot not assigned a cluster ID is assigned a cluster ID indicating awakening data (step S216). Total of 100 data plots each assigned a cluster ID are sorted in time series (step S218).

Sleep state is judged according to cluster IDs, which have been assigned to the sorted data plots (step S220). Specifically, sleep state during the detection time corresponding to data that is assigned cluster ID 1 is judged as NREM sleep. Sleep state during the detection time corresponding to data that is assigned cluster ID 2 is judged as REM sleep. Thus, clustering makes it possible to judge sleep state accurately.

When 120 minutes of cycle frames are set at setting intervals of one minute, a particular point of time is included in a plurality of cycle frames. In such a case, different judgments can be derived in the respective cycle frames for that particular point of time. From the point of view of real-time performance, it is desirable that the judgment be derived from a cycle frame in which the particular point of time is at the rearmost end thereof. In other words, after elapse of a predetermined period of time, sleep state at a particular point of time is judged based on a cycle frame that ends the particular point of time.

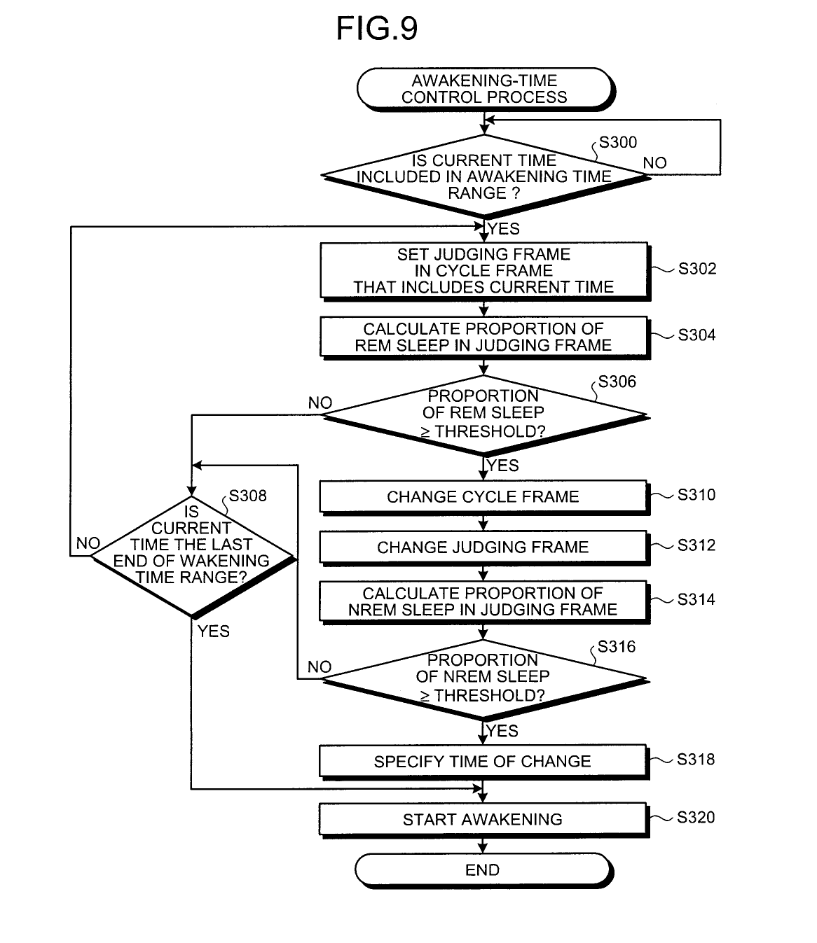

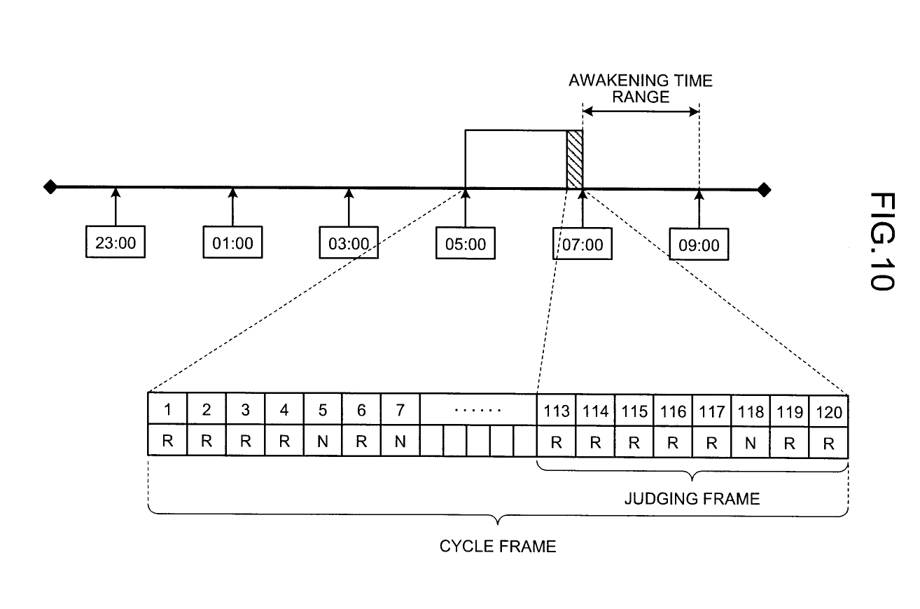

The awakening-time control process performed at step S160 of FIG. 7 is explained in detail with reference to FIG. 9. If the current time is within an awakening time range preset based on awakening time (Yes at step S300), the judging-frame setting unit 142 sets a judging frame in a cycle frame that includes the current time (step S302). If the current time is, for example, 07:00 o'clock, a judging frame of seven minutes from 06:53 o'clock to 07:00 o'clock is set in a cycle frame of 120 minutes ending at 07:00 o'clock. The change-timing detecting unit 146 calculates the proportion of REM sleep in the judging frame set by the judging-frame setting unit 142 (step S304). Sleep state is specified every one minute in the judging frame as shown in FIG. 10. Because the judging frame is of seven minutes, seven judgment results are obtained.

Referring back to FIG. 9, if the proportion of REM sleep is equal to or larger than a threshold from the judgment results in the judging frame (Yes at step S306), it is judged that the subject is currently in REM sleep and is yet to enter NREM sleep. The threshold is set in advance, for example, to 60%. The threshold can be arbitrary, and need not be limited to 60%.

If the proportion of REM sleep is smaller than the threshold from the judgment results in the judging frame (No at step S306), the judging-frame setting unit 142 judges whether the current time is the last end of the awakening time range or not (step S308). If the current time is the last end of the awakening time range (Yes at step S308), the awakening control unit 154 starts awakening process (step S320). On the other hand, if the current time is not the last end of the awakening time range (No at step S308), the process control returns to step S302 and the judging-frame setting unit 142 sets a judging frame.

After one minute, the cycle frame is changed (step S310), and the judging frame is also changed (step S312). That is, both the cycle frame and the judging frame are delayed by one minute. When a judging frame ending at 07:00 o'clock is set at step S302, a cycle frame and a judging frame are respectively set at steps S310 and S312 such that both the frames end at 07:01 o'clock.

The proportion of NREM sleep in the judging frame set at step S312 is calculated (step S314). If the proportion of NREM sleep is equal to or larger than the threshold (Yes at step S316), the change-timing detecting unit 146 specifies the current time as a time when sleep state has changed from REM sleep to NREM sleep (step S318). This threshold is identical to the threshold used at step S306. However, it is possible to set different thresholds to check the proportion of REM sleep and NREM sleep. The awakening control unit 154 starts awakening process (step S320).

If the proportion of NREM sleep is smaller than the threshold (No at step S316), i.e., the subject is judged to already be in REM sleep, the judging-frame setting unit judges whether the current time is the last end of the awakening time range or not (step S308). If the current time is not the last end of the awakening time range (No at step S308), the process control returns to step S302 and the judging-frame setting unit 142 sets the judging frame and the cycle frame.

Further, if all judgment results of step S306, S308 and S316 are YES, the starting awakening process (step S320) is eventually performed, and the awakening-time control process ends (step S160).

As another example, if the proportion of REM sleep is smaller than the threshold (No at step S306), it is also allowable to wait until the sleep state of the subject changes from NREM sleep to REM sleep. In this case, after the subject has entered REM sleep, timing of a change from REM sleep to NREM sleep is specified by the process from step S310 to step S318. Awakening is started at this timing.

As described above, with reference to changes in the proportion of NREM sleep and REM sleep in judgment frames, it is possible to accurately specify timing of a change from REM sleep to NREM sleep.

According to experiments conducted on different subjects, it is found that if a person is awakened at the timing of the change from REM sleep to NREM sleep, the person wakes up in a comfortable manner. Therefore, in the first embodiment, awakening is started at the time the sleep state has changed from REM sleep to NREM sleep. Thus, a person can be awakened in a comfortable manner.

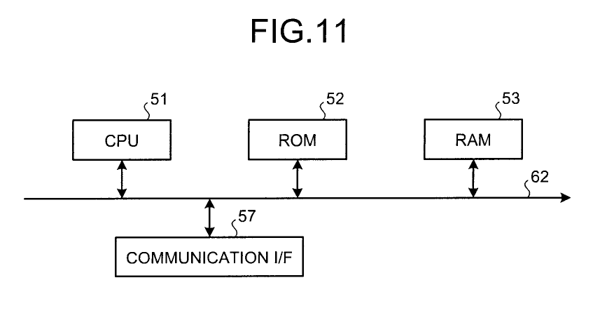

FIG. 11 is a block diagram of a hardware configuration of the autonomic-nervous state judging device 100. As shown in FIG. 11, the autonomic-nervous state judging device 100 includes a read-only memory (ROM) 52, a central processing unit 51, a random access memory (RAM) 53, a communication interface 57, and a bus 62. The ROM 52 stores therein computer programs such as a sleep state judging program for the sleep-state judgment process and executes the computer programs to perform various processes. The central processing unit 51 controls the autonomic-nervous state judging device 100 according to the computer programs stored in the ROM 52. The RAM 53 stores therein various types of data necessary to control the autonomic-nervous state judging device 100. The communication interface 57 is connected to a network for communication. The bus 62 connects each of the parts.

The sleep-state judgment program can be provided as being stored in a portable physical medium, such as a flexible disk (FD), a compact disc-read only memory (CD-ROM), and a digital versatile disk (DVD).

The sleep-state judgment program is read from the above-mentioned storage medium and is loaded into a main storing device of a computer to be executed thereon. Thus, each unit explained above can be implemented on the main storing device.

The sleep-state judgment program can be downloaded from another computer that is connected to the computer through a network such as the Internet, and executed by the computer.

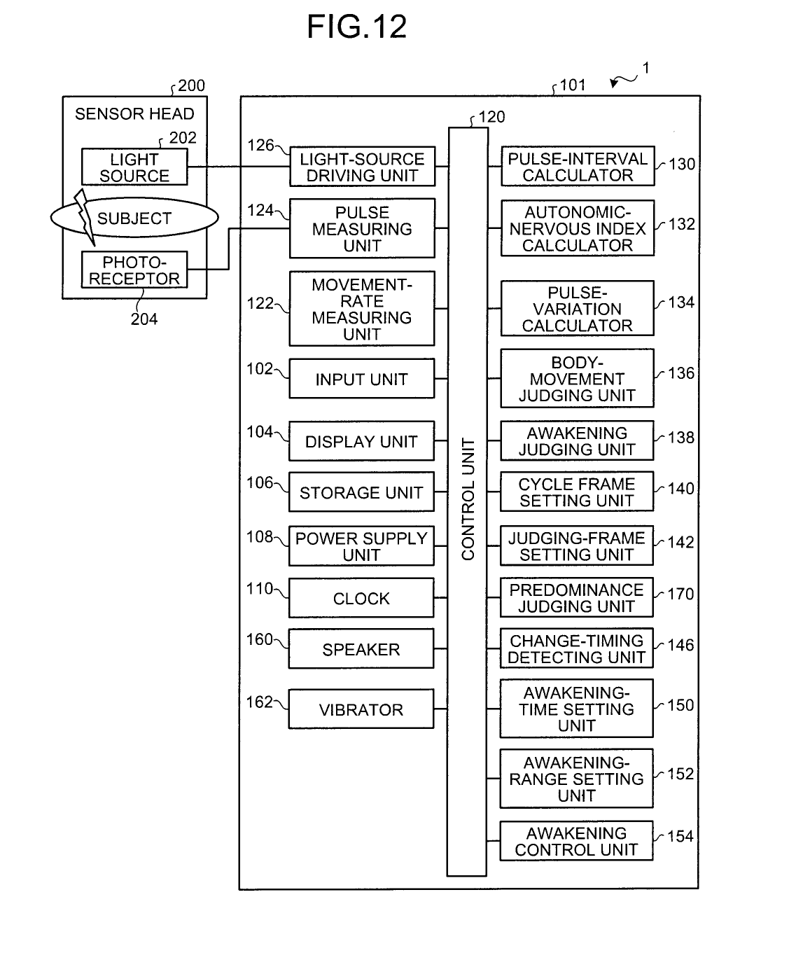

According to a second embodiment of the present invention, an autonomic-nervous state judging system 2 controls awakening based on sympathetic or parasympathetic predominance. As shown in FIG. 12, an autonomic-nervous state judging device 101 in the autonomic-nervous state judging system 2 includes a predominance judging unit 170 in place of the sleep-state judging unit 144.

The predominance judging unit 170 judges whether sympathetic or parasympathetic is predominant as the state of the autonomic nervous system based on the autonomic-nervous indices LF and HF and pulse variation. The awakening control unit 154 controls awakening based on the judgment by the predominance judging unit 170.

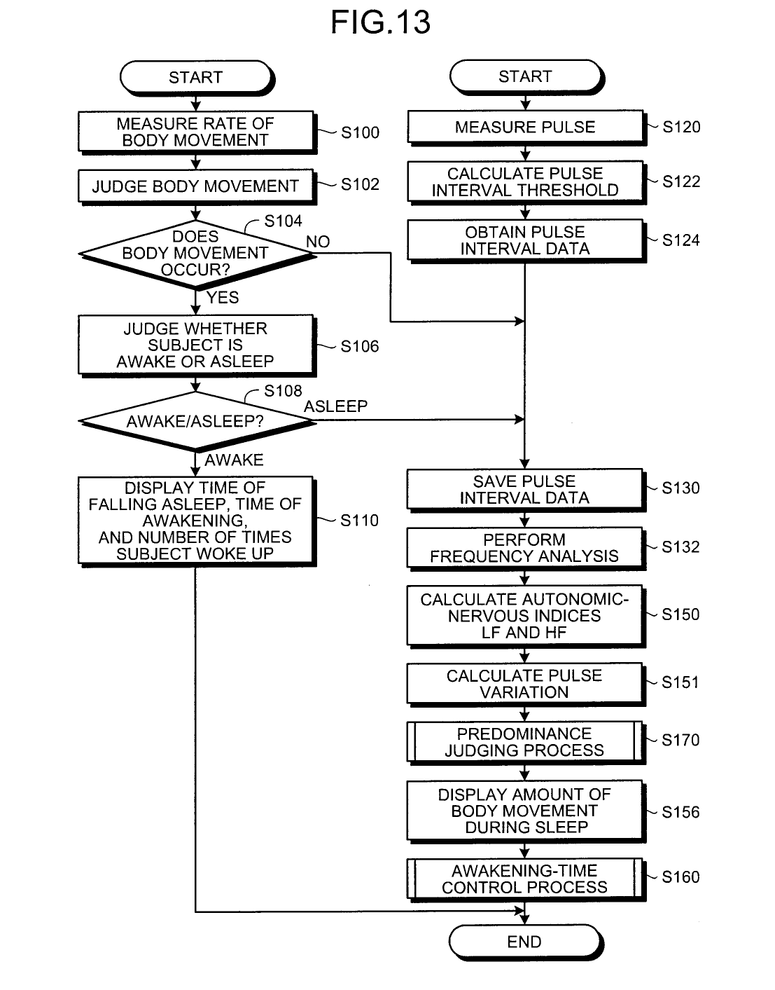

FIG. 13 is a flowchart of an awakening process according to the second embodiment. First, the autonomic-nervous index calculator 132 calculates the autonomic-nervous indices LF and HF, and the pulse-variation calculator 134 calculates pulse variation. Next, the predominance judging unit 170 performs a predominance judgment process to judge whether sympathetic or parasympathetic is predominant based on the autonomic-nervous indices LF and HF and the pulse variation (step S170). In the awakening-time control process (step S160), awakening is started based on the judgment by the predominance judging unit 170.

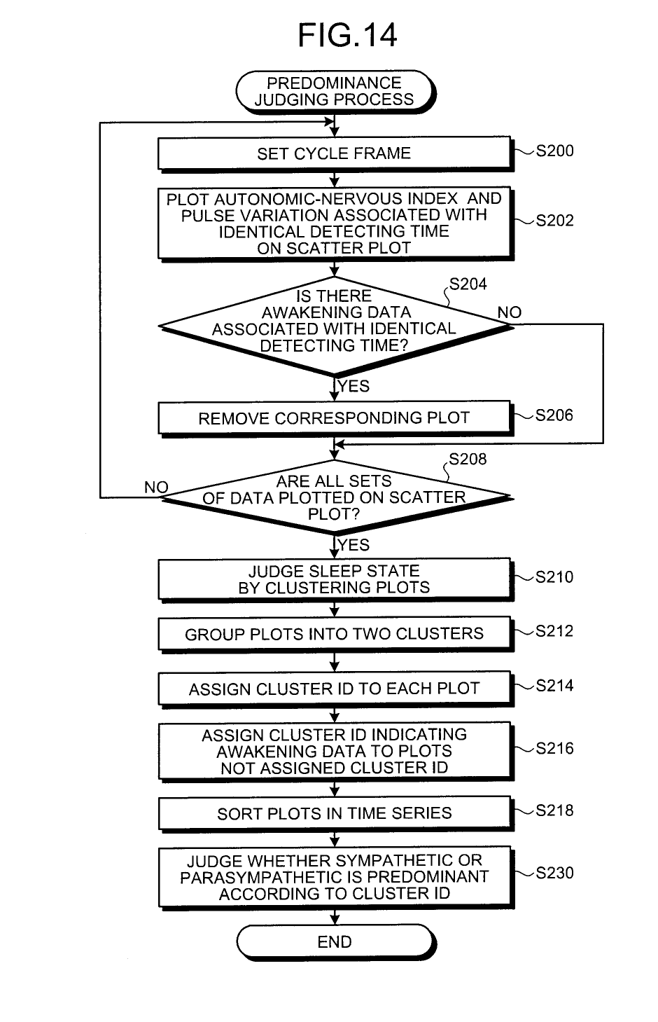

The predominance judgment process performed at step S170 of FIG. 13 is explained in detail with reference to FIG. 14. In the process from step S200 to step S218 in the predominance judgment process, data plots for each time unit are grouped into two clusters, and the data plots are each assigned a cluster ID. Incidentally, the process from step S200 to step S218 is identical to the process from step S200 to step S218 in FIG. 8 previously described for the sleep-state judgment process in the first embodiment.

At a detection time corresponding to data that is assigned cluster ID 1, it is judged that parasympathetic is predominant. At a detection time corresponding to data that is cluster ID 2, it is judged that sympathetic is predominant (step S230).

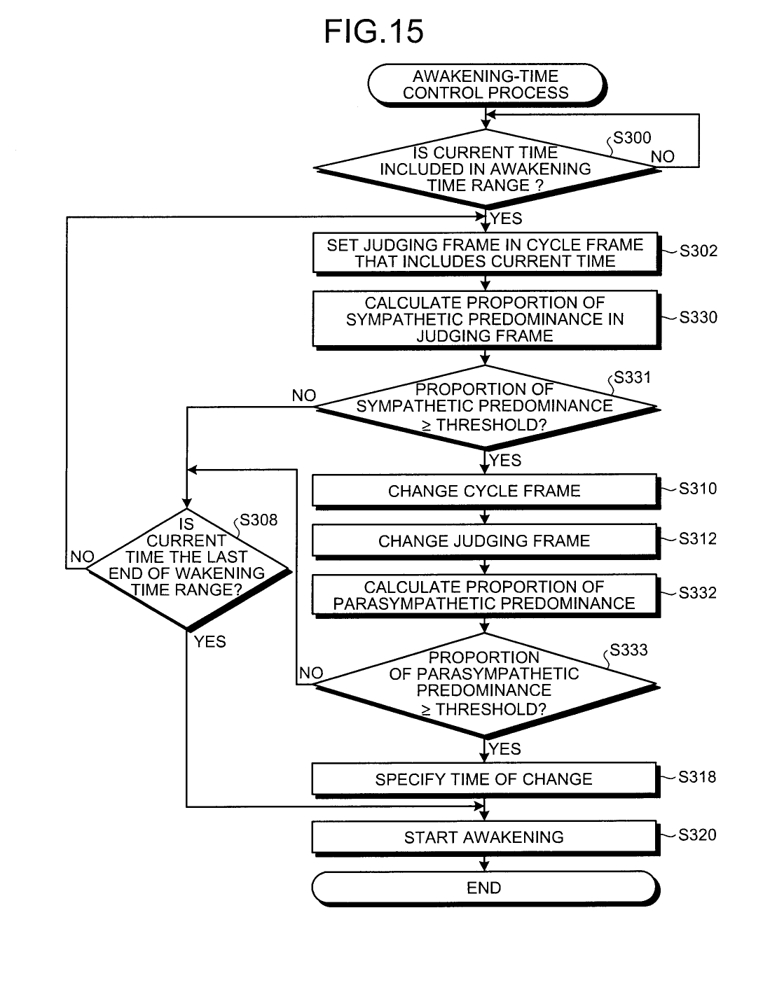

The awakening-time control process performed at step S160 of FIG. 13 is explained in detail with reference to FIG. 15. When the current time is within an awakening time range preset based on awakening time (Yes at step S300), the judging-frame setting unit 142 sets a judging frame in a cycle frame that includes the current time (step S302). The change-timing detecting unit 146 calculates the proportion of sympathetic predominance in the judging frame set by the judging-frame setting unit 142 (step S330).

If the proportion of sympathetic predominance is equal to or larger than a threshold (Yes at step S331), it is judged that sympathetic is currently predominant, and a shift from sympathetic to parasympathetic predominance has not occurred.

After one minute, a cycle frame is changed (step S310), and the judging frame is also changed (step S312). The proportion of parasympathetic predominance in the judging frame is calculated (step S332). If the proportion of parasympathetic predominance is equal to or larger than a threshold (Yes at step S316), the change-timing detecting unit 146 specifies the current time as a time at which a shift from sympathetic to parasympathetic predominance has occurred (step S318). The awakening control unit 154 starts awakening process (step S320), and the awakening-time control process ends (step S160).

The autonomic-nervous state judging system 2 has otherwise the same configuration and operates in a similar manner as the autonomic-nervous state judging system 1.

Incidentally, the above-described embodiments are susceptible of various modifications. For example, awakening time can be controlled by detecting the timing of a change from NREM sleep to REM sleep. In the same manner, awakening time can be controlled by detecting the timing of a shift from parasympathetic to sympathetic predominance.

In addition, audio output and a vibrator are cited for use in awakening a subject by way of example and without limitation. Instead, a subject can be awakened by using an electric current or a scent. A subject can also be awakened by plying music or by controlling light at awakening time. It is also possible to control the environment in which a subject is awakened by controlling air temperature, humidity, air pressure and the like. The environment can also be controlled by generating high concentration of oxygen to activate a subject. A subject can be stimulated with heat according to the season.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. An autonomic-nervous state judging device comprising:
a measuring unit that measures, at regular intervals, an autonomic-nervous index that indicates state of autonomic nervous system of a subject in a sleep state;
a first setting unit that sets a plurality of cycle frames of a first time length, each of which includes one sleep cycle, at intervals shorter than the first time length;
a second setting unit that sets a judging frame of a second time length shorter than the first time length at a predetermined position in each of the cycle frames;
a specifying unit that specifies state of autonomic nervous system in the judging frame based on the autonomic-nervous index in corresponding one of the cycle frames; and
a detecting unit that detects time of a change in the state of autonomic nervous system specified by the specifying unit.

2. The device according to claim 1, wherein the second setting unit sets the judging frame such that an end of the judging frame corresponds to a latest time in each of the cycle frames.

3. The device according to claim 1, wherein the specifying unit specifies the state of autonomic nervous system at different time points at predetermined intervals in the judging frame.

4. The device according to claim 1, wherein
the specifying unit specifies the state of autonomic nervous system based on continuity of predetermined state of autonomic nervous system in the judging frame, and
the detecting unit detects the time of a change in the state of autonomic nervous system based on a proportion of the predetermined state.

5. The device according to claim 4, wherein
the specifying unit specifies any one of non rapid eye-movement sleep state and rapid eye-movement sleep state as the state of autonomic nervous system based on continuity of the sleep state, and
the detecting unit detects time of a change from the non rapid eye-movement sleep state to the rapid eye-movement sleep state when a proportion of rapid eye-movement sleep in the judging frame exceeds a threshold.

6. The device according to claim 4, wherein
the specifying unit specifies any one of non rapid eye-movement sleep state and rapid eye-movement sleep state as the state of autonomic nervous system based on continuity of the sleep state, and
the detecting unit detects time of a change from the rapid eye-movement sleep state to the non rapid eye-movement sleep state when a proportion of non rapid eye-movement sleep in the judging frame exceeds a threshold.

7. The device according to claim 4, wherein
the specifying unit specifies any one of sympathetic predominance state and parasympathetic predominance state as the state of autonomic nervous system based on continuity of the predominance state, and
the detecting unit detects time of a change from the parasympathetic predominance state to the sympathetic predominance state when a proportion of sympathetic predominance in the judging frame exceeds a threshold.

8. The device according to claim 4, wherein
the specifying unit specifies any one of sympathetic predominance state and parasympathetic predominance state as the state of autonomic nervous system based on continuity of the predominance state, and
the detecting unit detects time of a change from the sympathetic predominance state to the parasympathetic predominance state when a proportion of parasympathetic predominance in the judging frame exceeds a threshold.

9. The device according to claim 1, further comprising a control unit that controls environment around a subject based on detected time of a change in the state of autonomic nervous system.

10. The device according to claim 9, wherein the control unit controls the environment by using at least one of light, sound, scent, temperature, humidity, pressure, electric current, voltage, heat, oxygen, and vibration.

11. An autonomic-nervous state judging method comprising:
measuring, at regular intervals, an autonomic-nervous index that indicates state of autonomic nervous system of a subject in a sleep state;
setting a plurality of cycle frames of a first time length, each of which includes one sleep cycle, at intervals shorter than the first time length;
setting a judging frame of a second time length shorter than the first time length at a predetermined position in each of the cycle frames;
specifying unit state of autonomic nervous system in the judging frame based on the autonomic-nervous index in corresponding one of the cycle frames; and
detecting time of a change in the state of autonomic nervous system specified at the specifying.

12. A computer program product comprising a computer usable medium having computer readable program codes embodied in the medium that, when executed, causes a computer to execute:
measuring, at regular intervals, an autonomic-nervous index that indicates state of autonomic nervous system of a subject in a sleep state;
setting a plurality of cycle frames of a first time length, each of which includes one sleep cycle, at intervals shorter than the first time length;
setting a judging frame of a second time length shorter than the first time length at a predetermined position in each of the cycle frames;
specifying unit state of autonomic nervous system in the judging frame based on the autonomic-nervous index in corresponding one of the cycle frames; and
detecting time of a change in the state of autonomic nervous system specified at the specifying.
